(19) United States
(12) Patent Application Publication
(10) Pub. No.: US 2015/0209887 A1
(43) Pub. Date: Jul. 30, 2015

(54) WELDING HELMET WITH HEADS UP DISPLAY

(71) Applicant: LINCOLN GLOBAL, INC., CITY OF INDUSTRY, CA (US)

(72) Inventor: Carmen DeLisio, Hudson, OH (US)

(21) Appl. No.: 14/682,340

(22) Filed: Apr. 9, 2015

Related U.S. Application Data
(63) Continuation-in-part of application No. 14/037,699, filed on Sep. 26, 2013.
Application No. 14/037,699 is a continuation of application No. 12/577,824, filed on Oct. 13, 2009, now Pat. No. 8,569,655.
(60) Provisional application No. 61/977,275, filed on Apr. 9, 2014.

(51) Int. Cl. B23K 9/095; B23K 9/32; G02B 27/01; A61F 9/06

(52) U.S. Cl. CPC B23K 9/0953; A61F 9/06; B23K 9/322; G02B 27/017

(57) ABSTRACT

A welding system and welding helmet is provided, where the welding helmet is capable of providing an image representative of information from an associated welding operation where the image appears as a head-up display (HUD) in the welding helmet. The helmet displays the information at a focal point which coincides with a working distance of a welding operation so that a user need not change his/her focus during a welding operation.

### PRIORITY AND INCORPORATION BY REFERENCE

This application is a continuation-in-part of U.S. application Ser. No. 14/037,699, filed Sep. 26, 2013, which is a continuation of U.S. application Ser. No. 12/577,824, filed on Oct. 13, 2009, which issued as U.S. Pat. No. 8,569,655, the specifications of which are incorporated herein by reference in their entirety. The present application also claims priority to U.S. Provisional Patent Application No. 61/977,275, which is incorporated herein by reference in its entirety.

### BRIEF SUMMARY OF THE INVENTION

### Field of the Invention

This invention relates in general to equipment used in welding. Devices, systems, and methods consistent with the invention relate to the monitoring of welding parameters and specifically to a welding helmet with a head up display (HUD).

### DESCRIPTION OF THE RELATED ART

Welding is an important process in the manufacture and construction of various products and structures. Applications for welding are widespread and used throughout the world, for example, the construction and repair of ships, buildings, bridges, vehicles, and pipe lines, to name a few. Welding may performed in a variety of locations, such as in a factory with a fixed welding operation or on site with a portable welder.

In manual or semi-automated welding a user/operator (i.e. welder) directs welding equipment to make a weld. For example, in arc welding the welder may manually position a welding rod or welding wire and produce a heat generating arc at a weld location. In this type of welding the spacing of the electrode from the weld location is related to the arc produced and to the achievement of optimum melting/fusing of the base and welding rod or wire metals. The quality of such a weld is often directly dependent upon the skill of the welder.

Welders generally rely upon a variety of information when welding. This information includes, for example, current and voltage. Traditionally, welders would need to look at gauges on the control panel of the welding equipment to gain this information. This would require the welder to direct their field of vision away from the welding work area and as such was undesirable. In addition, in many cases, the welding machine may not be located close to the work space. In such cases, the welding machine is operated by a cable-connected remote control that can be used to change parameters such as, e.g., welding power, polarity, arc characteristics, etc. However, before the process can be set up, the welder may need to see the display readouts that are physically located on the machine. The setting-up process may require many trips before the set-up is completed.

In the past, efforts have been made to provide welders with information during welding, such as in the method disclosed in U.S. Pat. No. 4,677,277, where current and voltage are monitored to produce an audio indication to the operator as to the condition of the arc in arc welding. However, monitors consisting only of audio arc parameter indicators are hard to hear and interpolate and are not capable of achieving the desired closeness of control and quality of weld often required.

More recently, as disclosed in U.S. Pat. No. 6,242,711, an apparatus for monitoring arc welding has been developed that provides a welder with real-time voltage and current conditions of the welding arc where information in the form of lights, illuminated bar graphs, light projections, illuminated see-through displays, or the like are placed within the visual range of the helmet wearing operator and located in proximity to the helmet viewing window in the helmet. However, in this apparatus a welder must still move their visual focus away from the welding work area in order to focus on the information located proximate to the welding window or the welder must accept the information peripherally while continuing to focus on the welding work area.

### BRIEF SUMMARY OF THE INVENTION

This invention relates to a welding helmet that is capable of providing an image representative of information from an associated welding operation where the image appears as a head-up display (HUD) in the welding helmet.

Various aspects will become apparent to those skilled in the art from the following detailed description and the accompanying drawings.

### BRIEF DESCRIPTION OF THE DRAWINGS

The above and/or other aspects of the invention will be more apparent by describing in detail exemplary embodiments of the invention with reference to the accompanying drawings, in which:

FIG. 1 is a schematic view of a welding system according to the present invention;

FIG. 2 is an enlarged view of a welding helmet similar to the helmet of FIG. 1 including a camera;

FIG. 3 is a cross-sectional diagram of a welding helmet similar to the helmet of FIG. 2 including a projector;

FIG. 4 is a cross-sectional diagram of a welding helmet similar to the helmet of FIG. 3 including an integrated video display;

FIG. 5 is a perspective view of a welding helmet similar to the helmet of FIG. 2 including binocular cameras;

FIG. 6 is an interior view of a welding helmet similar to the helmet of FIG. 5 showing binocular viewing screens;

FIG. 7 is a cross-sectional diagram of an exemplary embodiment a welding helmet with a HUD;

FIG. 8 is a schematic view of a welding system according to an exemplary embodiment of present invention;

FIG. 9 is an interior view of a welding helmet with a HUD;

FIGS. 10A and 10B are an interior views of a welding helmet with a HUD;

FIG. 11 is a cross-sectional diagram of an exemplary embodiment of a welding helmet with a HUD;

FIG. 12 illustrates exemplary views of information that can be displayed.

### DETAILED DESCRIPTION OF EXEMPLARY EMBODIMENTS

Exemplary embodiments of the invention will now be described below by reference to the attached Figures. The described exemplary embodiments are intended to assist the understanding of the invention, and are not intended to limit the scope of the invention in any way. Like reference numerals refer to like elements throughout.

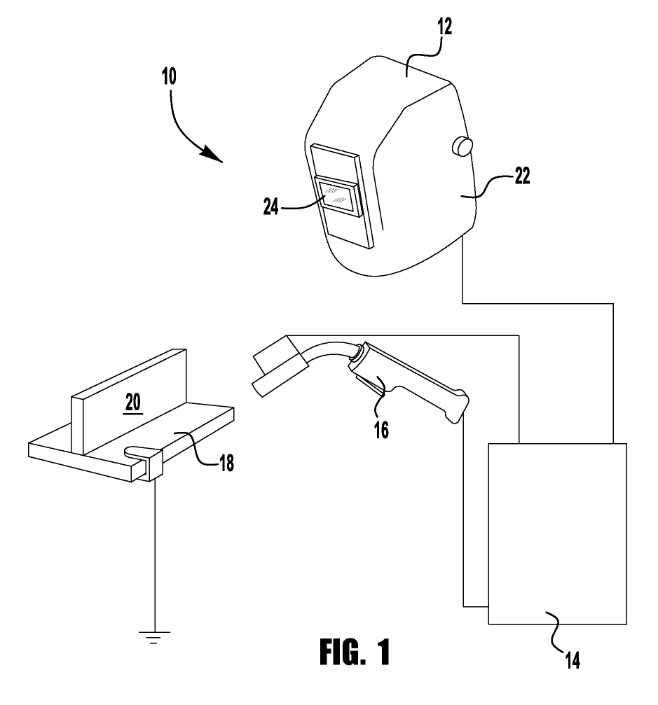

Referring now to the drawings, there is illustrated in FIG. 1 a welding system 10. The welding system 10 includes a welding helmet 12, a welding system 14, a welding gun 16 and a work piece 18. The work piece 18 generally defines a welding work area 20 where the welding gun 16 may be used to form a weld.

The welding system 14 includes welding equipment for generating a welding current and voltage, a welding control system for controlling the welding current and voltage, and a monitoring system for monitoring the welding current and voltage. That is, the welding system, can be on known or used welding power supply having a known construction and operation. The monitoring system may also monitor a variety of other operating parameters, such as but not limited to, wire feed speed, amount of wire used/amount of wire remaining, any type of welding feedback desired by the operator and any other desired operating parameter.

The welding helmet 12 includes a main body 22 with a visual display 24 connected to the main body 22. The display 24 may be a window including a welding lens, a video monitor, such as an LCD display or LED array, or any other device suitable to allow a welder to see the welding work area 20. It must be understood that in such an example where the display 24 is a video monitor video processing may be utilized to enhance the pictures of the welding operation. Further, recording devices may optionally be included in the display, for example, to record and later playback welding operations for analysis and/or evaluation.

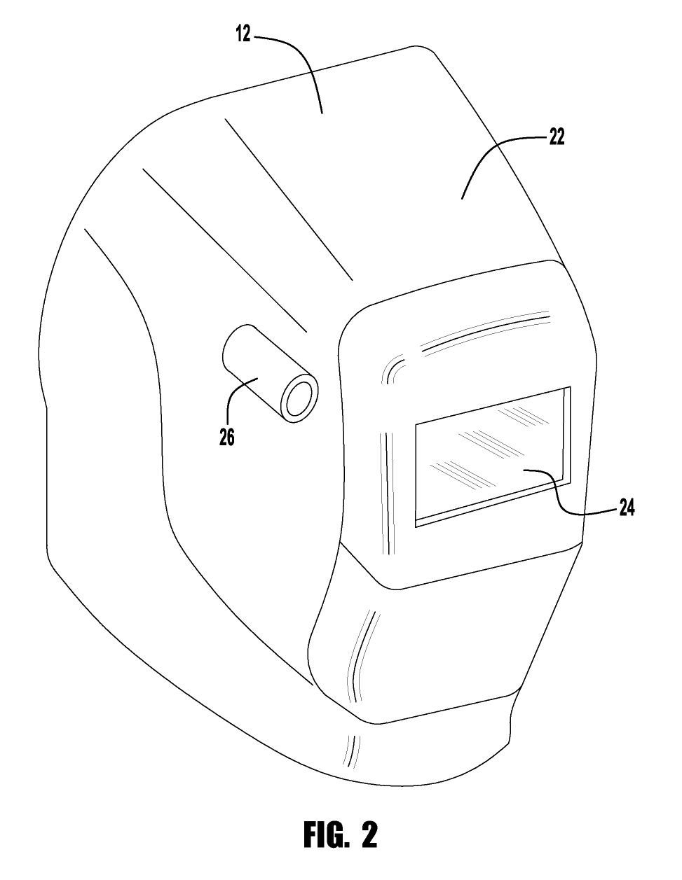

As shown in FIG. 2, a welding helmet 12 may include a camera 26 mounted at or proximate to the point of view of the welder. In the example where the visual display 24 is a video monitor, the camera 26 may provide video pictures of the welding work area 20 to the display 24. Further, the camera 26 can be used to record the welding operation as it is ongoing, so that the welding operation can be viewed at a later time.

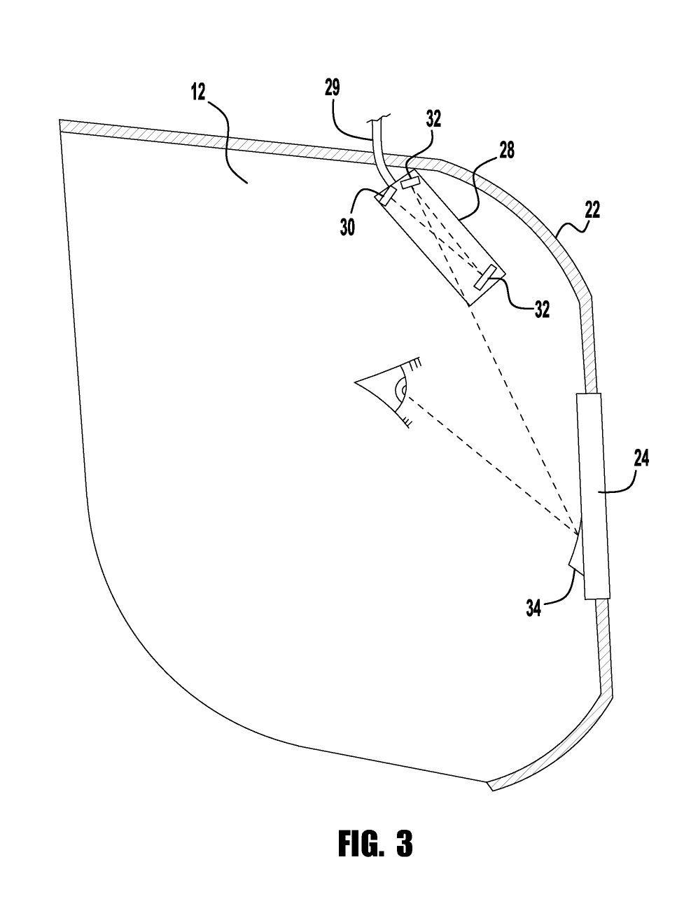

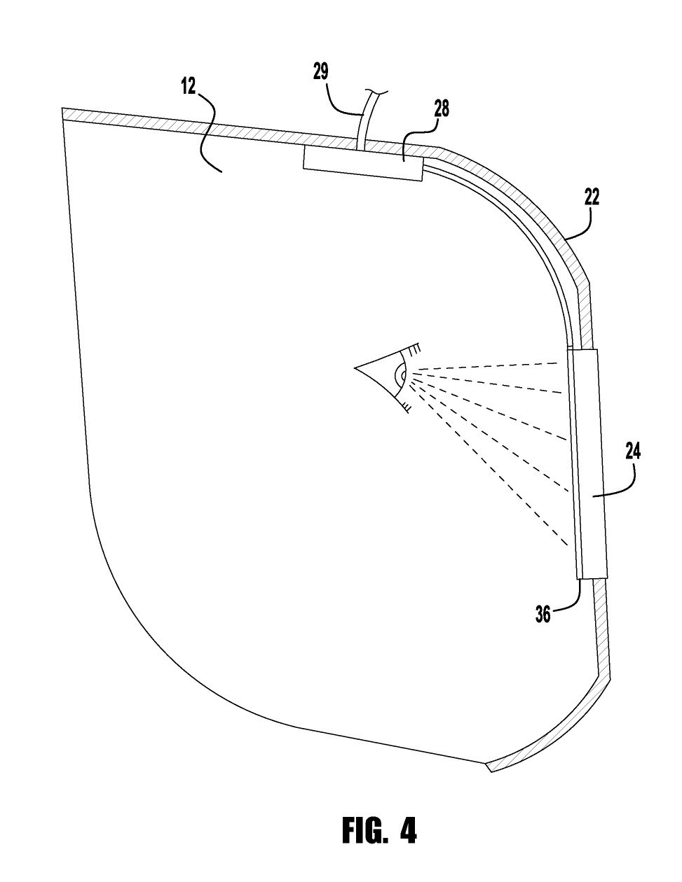

As shown in FIGS. 3 and 4 an information generating mechanism 28 is in communication with the monitoring system of the welding system 14 and capable of generating an image representative of information from the monitoring system based upon the monitored welding parameter, such as current and voltage upon the visual display 24 where the focus of the image is at a focus range (i.e., having a focal point) with an associated welding work area, e.g. outside of the main body 22 of the welding helmet 12. That is, the focal range of the image displayed in/on the display 24 is set to be at a range associated with the location of the welding work area 20. For example, the image may be symbolic, alpha-numeric, or any other device suitable to indicate the information. Thus, a welder may view an image representative of information about a welding operation without removing focus from the work area. Thus, in at least one embodiment the welder may focus on the work area and the image of information at the same time.

It must be understood that among other types of information, along with a variety of other parameter, the information based upon welding current and voltage includes, but is not limited to, welding current feedback, welding voltage feedback, control settings of the welding equipment, statistical information of the welding process, benchmarks or limits including capacity representations, alerts including material shortage or low flow, a representation of an intended or desired weld, etc.

Further, in one embodiment, the camera 26 is used to calibrate the depth of the image relative to the welding work area 20. This calibrated depth can be used to determine the focus of the information displayed on the display 24. For example, if the camera 26 determines that the distance from the helmet to the work area is 2 feet, the images and/or information shown on the display 24 is displayed such that the image has a focal point which would be at 2 feet beyond the helmet. As explained above, this allows the displayed information to be displayed at a same focal length as the weld area 20 so that the welder need not change his/her eye focus during a welding operation. In another embodiment, positions sensors on the welding gun may be used to calibrate the depth of the image. Such sensors can include, but are not limited to, magnetic sensors, optical sensors, acoustics sensors, and the like, which are sensed using an appropriate sensing system to allow for the positioning of the welding gun to be determined. This data can be used to aid in determining the focal range/distance of the work area relative to the helmet. In particular applications it is highly desirable to carefully align the image and the welding work such that the information represented in the image is easy for the welder to access and such that the information in the image is readily accepted by the welder.

In the example where the visual display 24 is a video monitor, information generating mechanism 28 may include an image representative of information from the monitoring system based upon the monitored parameter, such as welding current and voltage, in video pictures of the welding work area 20 shown on the display 24.

As indicated at 29, the information generating mechanism 28 may be in wired or wireless communication with other devices as desired.

In FIG. 3, the information generating mechanism 28 is a projector. The projector may, for example, include an internal LCD display or LED array 30 along with a number of associated mirrors 32 to reflect the image generated to the visual display 24. The reflected image gives the image the appearance of depth relative to the visual display 24 and thus puts the image at a focus range with an associated welding work area and outside of the main body 22 of the welding helmet 12 and optionally at the same focal distance as the associated welding work area 20. Optionally, a reflective surface 34 may be placed upon a portion of the visual display 24 in order to achieve a desired amount of reflection or reflection angle. In one embodiment, teleprompter type technology may be utilized to place the image upon the display 24 or surface 34. Additionally, it must be understood that one embodiment includes the use of an LCD display or other similar display within the helmet to generate the image which is then sent along an optical path, such as by reflection or fiber optics or any other suitable device to place the image display 24 or surface 34.

In FIG. 4, the information generating mechanism 28 includes a screen, film, or sheet 36 integrated into the visual display 24. The sheet 36 may be a semi-transparent LCD film, electro-optic film, or any other suitable medium for the information generating mechanism 28 to produce an image generated in the visual display 24. In one application, the information generating mechanism 28 may project a stereogram on the welding lens such that a welder's eyes will separately view the images to create the perception of depth and thus focus the image at a focus range with the associated welding work area 20 and outside of the main body 22 of the welding helmet 12.

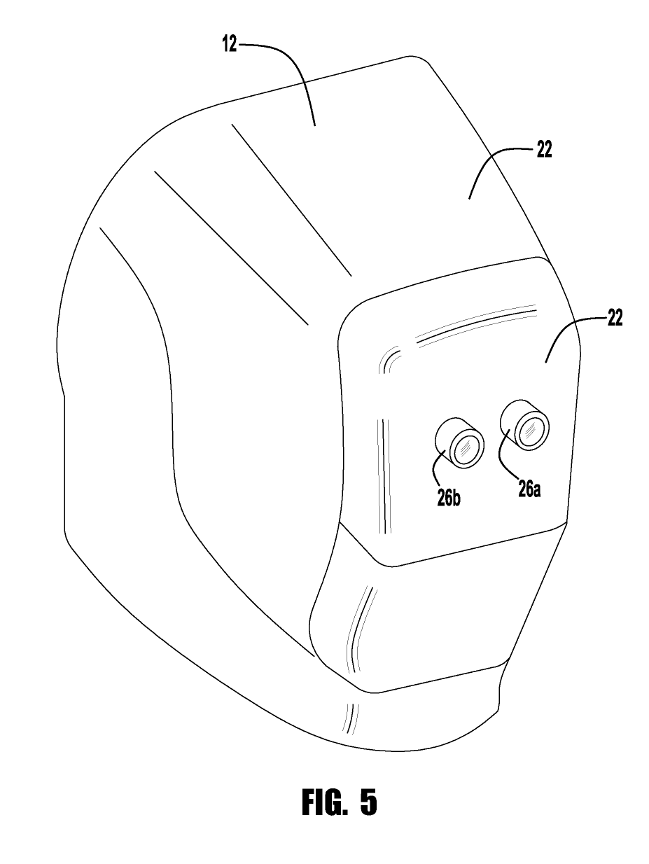

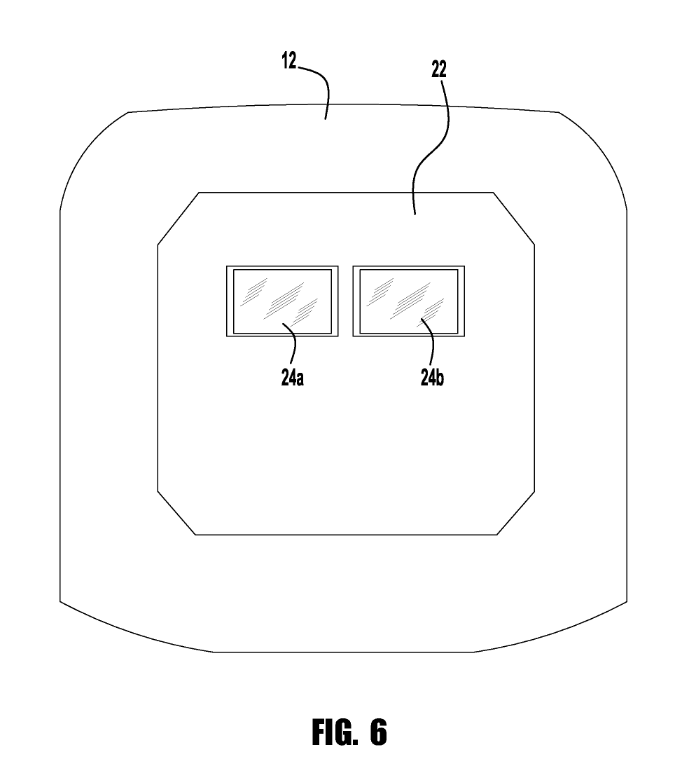

There is shown in FIG. 5 a welding helmet 12 including binocular cameras 26a and 26b. As shown in FIG. 6, these cameras 26a and 26b correspond to binocular viewing screens 24a and 24b. An information generating mechanism may produce an image to be generated in either of the viewing screens 24a or 24b or both. In one embodiment, the cameras 26a and 26b are placed in alignment with the screens 24a and 24b except on opposite sides of the main body 22, thus giving the welder the view directly in front of them. Additionally, in the embodiment with binocular cameras 26a and 26b and binocular viewing screens 24a and 24b the perception of depth of field is produced.

In any case, the image may be an overlay of text or graphics or video feedback. Additionally, it is contemplated that in at least one embodiment the system described above may be used in a remote welding situation, including but not limited to robotic welding or underwater welding.

While principles and modes of operation have been explained and illustrated with regard to particular embodiments, it must be understood, however, that this may be practiced otherwise than as specifically explained and illustrated without departing from its spirit or scope.

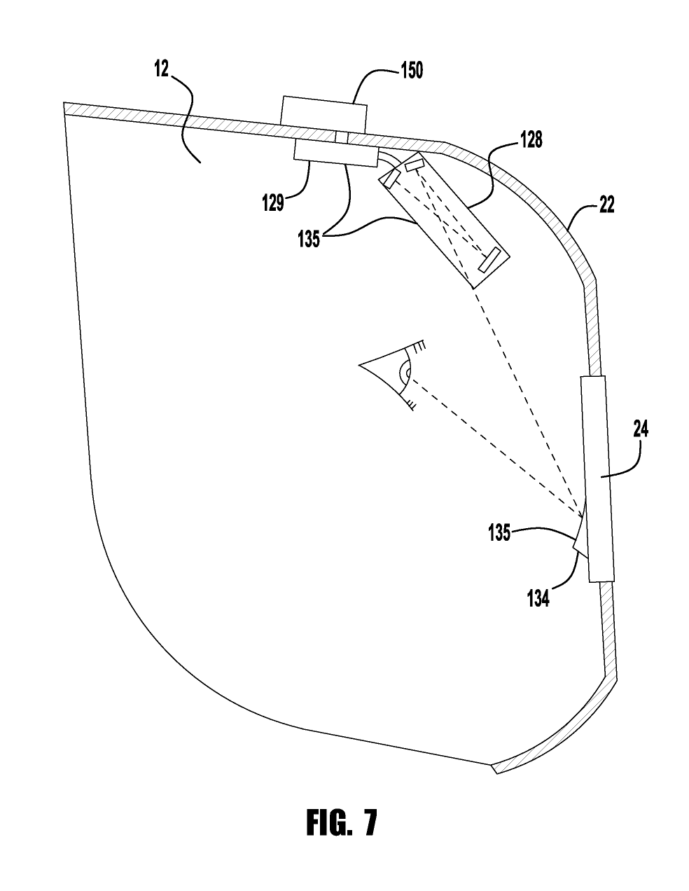

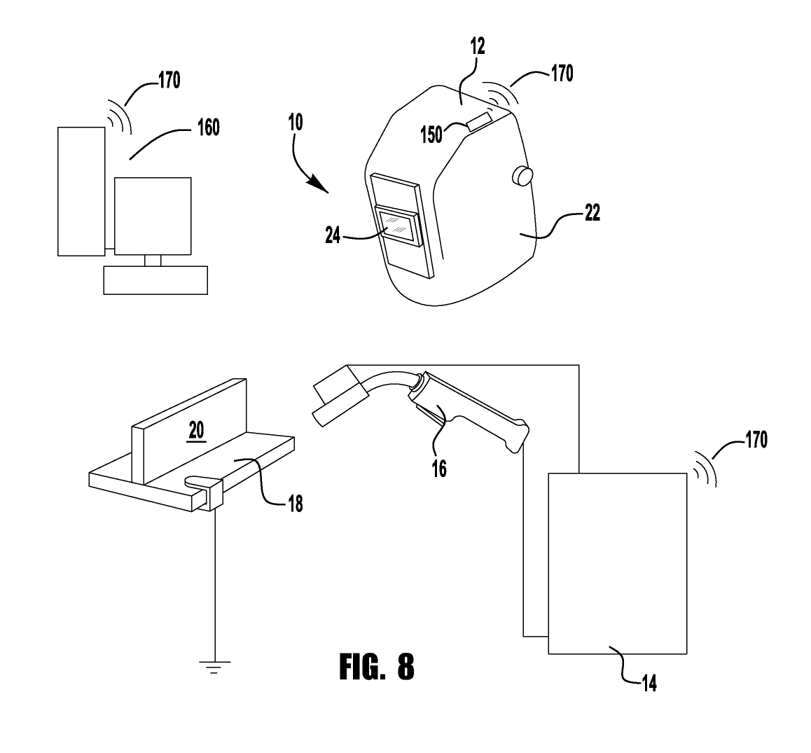

Some exemplary embodiments of the present invention, as illustrated in FIG. 7, include a welding helmet 12 with a head-up display (HUD) 135 for the welder. In some embodiments, the HUD 135 includes a projector 128, a combiner 134, and an information generating device 129. The projector 128 can be, e.g., an LED array, an LCD display, a laser, a combination LED/LCD system, or some other suitable projector system. The projector 128 projects an image onto the combiner 134. The image can be in the form of text, graphics, video, etc. The projector 128 receives image information, e.g., in the form of a digital signal, from information generating device 129. The information generating device 129 generates and/or processes the image based on information received from an external source such as, e.g., a welding system 14 or a computer system 160 (see FIG. 8). This information can include, among other types of information, welding parameters such as input power, input current, input voltage, welding voltage, welding current, welding power, tip-to-work distance, arc length, wire feed speed, etc. In some embodiments, the projector 128 and information generating device 129 can be integrated into a single physical unit. In some embodiments, the computer system 160 and/or the welding system 14 generates and/or processes the image and transmits the image information directly to projector 128, which can include and/or is connected to a wireless communication device.

The combiner 134 reflects the image projected from projector 128 to the welder. In some embodiments, light transmitted through lens 24 is also transmitted through combiner 134. Thus, the welder will see both the projected image and the field of view behind the combiner 134 at the same time. The light transmitted through lens 24 can be that from a welding arc transmitted through lens 24. In some embodiments, the lens 24 is of a type that rapidly and automatically changes from transparent to dark when the lens 24 detects that a welding arc has been initiated. The auto-darkening feature protects the welder's eyes from damage that could occur if the eye is exposed to the welding arc. The auto-darkening lens is transparent when no arc is detected and thus allows the welder to see the work space even when the welding helmet 12 is flipped down over the welder's face. With an auto-darkening lens, the light transmitted through lens 24 and combiner 134 can be either the light from the welding arc or normal room lighting depending on whether a welding operation is taking place.

In some embodiments, the combiner 134 collimates the reflected image such that the projected image appears to be at optical infinity. Thus, the welder will not have to re-focus to see both the work space and the projected image—even during the welding process. In some embodiments, the combiner 134 is an appropriate transparent material, e.g., a flat piece of glass, that is angled such that the projected image from the projector 128 is reflected to the welder as illustrated in FIG. 7. In some embodiments, the mounting of the combiner 134 to welding helmet 12 and/or lens 24 is such that the angle of reflection can be adjusted by the welder, as desired.

In some embodiments, the combiner 134 includes a coating that reflects monochromatic light from the projector 128. For example, the coating on the combiner 134 can be such that only, e.g., green light is reflected and all other light is transmitted through. Thus, the HUD 135 will provide the welder a transparent display that allows the welder to see information on the combiner 134 in green while still allowing the welder to view the work space. Of course, other coatings that reflect other colors or even multiple colors can be used on the combiner 134. For example, the combiner 134 can be coated such that it reflects the colors green and red. While in the normal operating range, the information, e.g., welding current, may be displayed in green and when outside the normal operating range, the information, e.g., welding current, can be displayed in red. The information provided to the welder can include welding operating parameters such as, e.g., input current, input voltage, input power, welding current, welding voltage, wire feed speed, contact tip-to-work distance, arc length, mode of operation, etc.

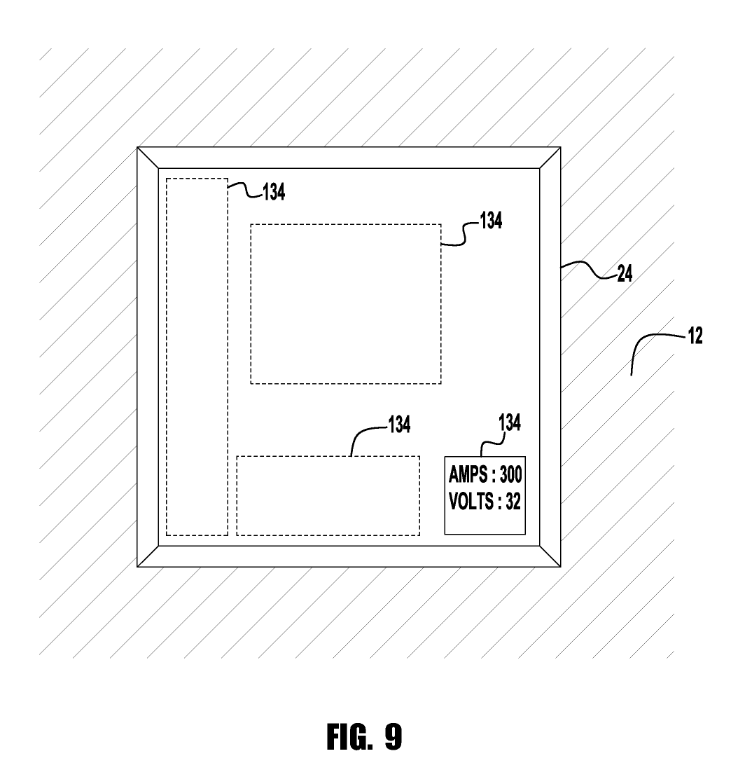

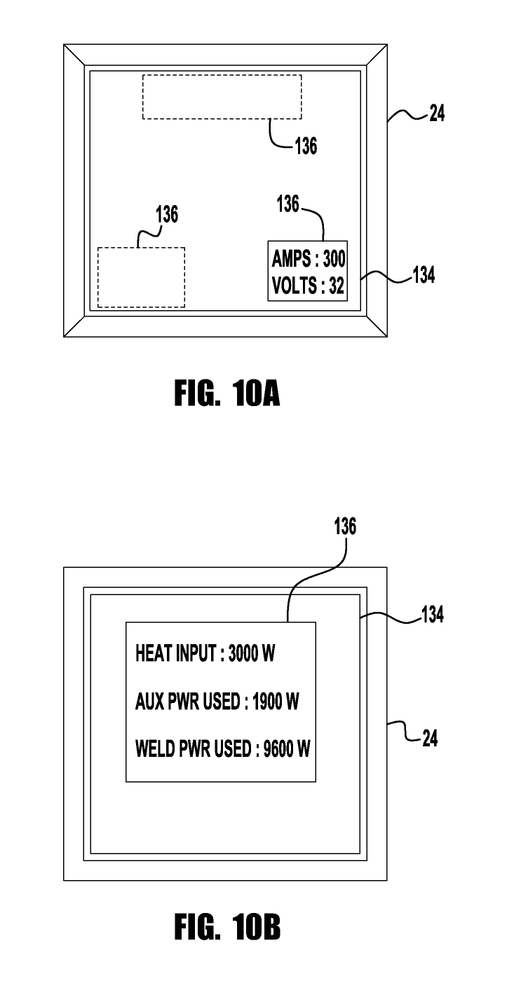

The size, shape, and placement of the combiner 134 relative to the lens 24 can vary, as desired. For example, FIG. 9 illustrates various sizes, shapes, and locations for the combiner 134. The illustrated sizes and locations are exemplary and any appropriate size, shape, and location can be utilized. For example, in some embodiments, the combiner 134 is sized such that it covers the entire opening of lens 24 (see FIGS. 10A and 10B). Similarity, an image window, i.e. the window in which actual information is displayed, on the combiner 134 can be sized and/or located as desired. For example, as seen in FIG. 10 A, during an actual welding process an image window 136 can be displayed in a corner and/or along an edge of the combiner 134 so that the welder is not distracted but still has the information available, if desired. When the welding process is not occurring, the image window 136 can be displayed larger as seen in FIG. 10B. The HUD 135 can be configured such that the image 136 is automatically resized based on whether welding system 14 is performing a welding operation. Alternately, or in addition, the HUD 135 can be configured such that the resizing of image 136 is a manual operation by the welder.

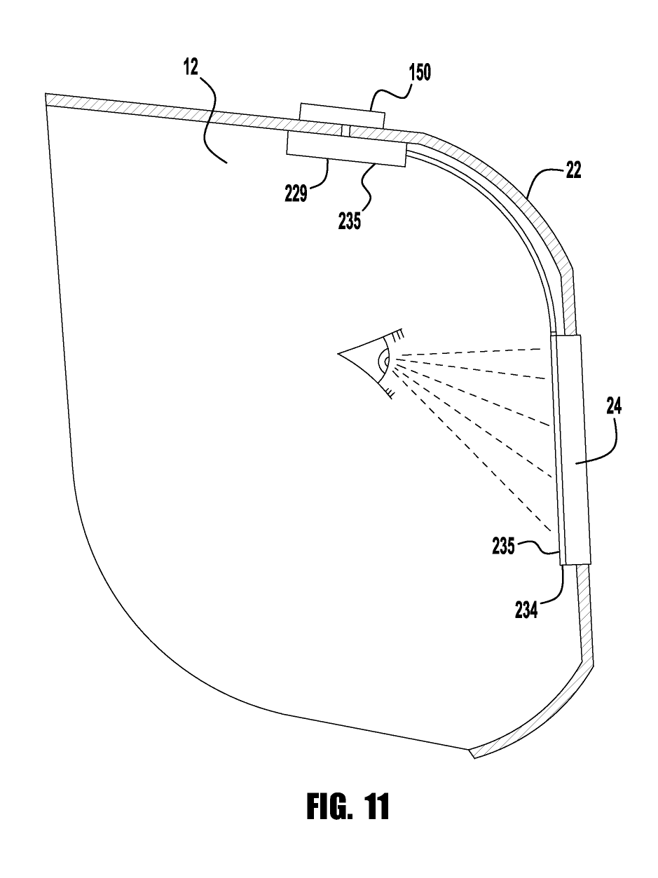

In some embodiments, the projector is not used. As illustrated in FIG. 11, a HUD 235 includes combiner 234 and information generating device 229. In this exemplary embodiment, the image is produced directly in the combiner 234. The combiner 234 can be, e.g., an LCD display, optical waveguide, an electro-optical medium, or some other suitable medium for producing an image. Similar to combiner 134 discussed above, the size, shape, and placement of the combiner 234 relative to the lens 24 can vary, as desired, including having a display size that is equal to the size of the window of lens 24. In addition, similar to image window 136 discussed above, an image window on combiner 234 can be sized and/or located as desired.

The combiner 234 receives image information, e.g., in the form a digital signal, from information generating device 229, which generates and/or processes the image based on information received from welding system 14 and/or computer system 160. In some embodiments, the combiner 234 and information generating device 229 can be integrated into a single physical unit. In some embodiments, the combiner 234 and lens 24 can be integrated into a single physical unit. In some embodiments, the combiner 234, information generating device 229, and lens 24 can be integrated into a single physical unit. In some embodiments, the computer system 160 and/or the welding system 14 generates and/or process the image and transmits the image information directly to combiner 234, which can include or is connected to a wireless communication device.

The information generating devices 129 and 229 can each include a communication device 150 to communicate via, e.g., a wireless network 170 or a wired network with welding system 14 and/or computer system 160. The wireless network 170 can operate using, e.g., Bluetooth, WiFi (IEEE 802.11) or some other wireless protocol. In some embodiments, the welding system 14 can provide information such as e.g., input power, input current, input voltage, welding current, welding voltage, welding power, contact tip-to-work distance, arc length, wire feed speed, etc. in real-time to, e.g., aid the welder while the welding operation is going on. Alternatively, or in addition, the welding system 14 can send welding performance information after the welder has stopped welding. For example, the welding system 14 can transmit information such as, e.g., heat input, duration of welding, etc. after, e.g., the welder system 14 is turned off, indicating that the welder is done welding. Such information might be useful to the welder in order to make corrections before starting the next welding segment.

In some embodiments, the computer system 160 performs all the calculations such as, e.g., heat input, welding duration, etc. The computer system 160 can communicate with the welding system 14 and/or the welding helmet 12 via, e.g., wireless network 170 or a wired network. In some embodiments, the computer system 160 collects, stores, and/or analyzes information received from the welding system 14. In some embodiments, the computer system 160 transmits the image information to the welding helmet 12 instead of or in addition to the welding system 14. In some embodiments, the computer system is incorporated into or is integral to the welding system 14.

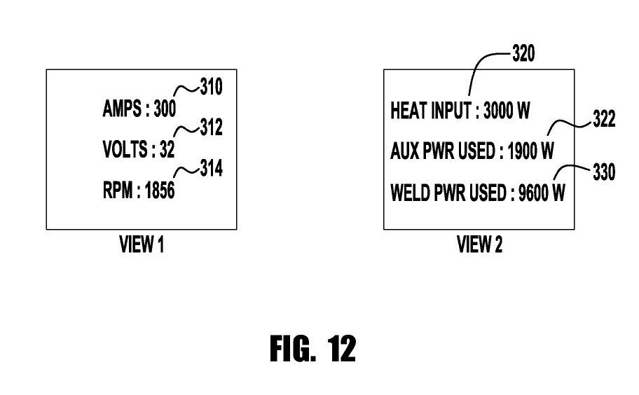

In some embodiments, the image information seen by the welder is configurable. For example, the computer system 160 and/or the welding system 14 can be configured with different “views” or image screens that the welder can select. For example, as illustrated in FIG. 12, the welding information can be presented to the welder using several image screens or “views.” View 1 can represent real-time operating parameters of an engine-driven welder such as, e.g., welder output amps 310, welder output volts 312, engine speed 314, etc. A second “view,” View 2, can represent performance totals such as, e.g., welding heat input 320, Auxiliary Power Used 322, Welding Power Used 324, etc. In other exemplary embodiments, other data can be shown. For example, the HUD can display engine related information such as RPM, engine temperature, oil pressure, air compressor output pressure (if equipped), and any trouble codes from an engine control computer to allow the welder to be warned of any issues. The views can be customized to meet the welder's needs. For example, the views can be customizable based on the type of welding (TIG, MIG, etc.), material being welded (steel, aluminum, etc.), type of weld (fillet, butt joint, etc.), or on some other basis. In addition, the views can be customized for each welder. For example, after a welder identifies himself or herself by, e.g., logging into the computer system 160 or welding system 14, by using a token such as, e.g., a RFID tag, or by some other means, the computer system 160 and/or the welding system 14 can display a set of “views” that are specific to the welder, e.g., based on the welder's preferences, experience level, etc.

The welder can turn the HUD 135, 235 on and off and scroll through the “views” using controls (not shown) located on the welding helmet 12. Alternatively, or in addition, the welder can control the HUD 135, 235 using voice commands. The welding helmet 12 can include a microphone system 140 (see FIG. 7) that picks up audio command from the welder. The microphone system 140 can then relay the audio commands to welding system 14 and/or computer system 160 using communication device 150. The welding system 14 and/or the computer system 160 interprets the commands and sends the appropriate instructions and information to the information generating device 129, 229. Information generating device 129, 229 will then control projector 128 and/or combiner 234 based on the received information and instructions. For example, a welder can say “SHOW CURRENT” and the system will display the welding current. Other voice commands can be used to allow the user to display the desired information. These voice commands can be used, prior to, during or after the completion of a welding process. In addition to the commands discussed above, the welder can adjust the size and location of the image window 136, the brightness of the image display, the color of the image display, etc. In some embodiments, the welder can control the opacity of the image to make the image more or less transparent, e.g., from nearly 100% transparent to 100% opaque. Along with welder adjustments, some display parameters such as, e.g., brightness, opacity, color, etc. can be adjusted automatically by at least one of information generating device 129, 229, computer system 160 and welding system 14 based on whether the welding arc is sensed and/or the level of ambient light in the room.

While the invention has been particularly shown and described with reference to exemplary embodiments thereof, the invention is not limited to these embodiments. It will be understood by those of ordinary skill in the art that various changes in form and details may be made therein without departing from the spirit and scope of the invention as defined by the above embodiments.

What is claimed is:

1. A welding helmet, comprising:
a main body portion;
a visual display portion which is coupled to the main body portion, where said display portion allows a welding operation to be viewed with said display portion;
an information generating mechanism which generates an image to be displayed on said visual display portion said that said image is viewable during said welding operation; and
a wireless communication device coupled to said information generating mechanism which receives information to be displayed in said image;
wherein said image is generated by said information generating mechanism is generated having a visual focal range which is located beyond said visual display portion at a position outside of said main body portion and wherein said focal range is correlated to a work area for said welding operation.

2. The welding helmet of claim 1, further comprising a camera mounted to an outside of said main body portion so that said camera can view the work area and sense a distance to said work area and said camera is used to calibrate said focal range of said image.

3. The welding helmet of claim 1, further comprising a camera mounted to an outside of said main body portion so that said camera can view the work area and said camera is used to record a welding operation in said work area.

4. The welding helmet of claim 1, wherein said image includes information related to said welding operation.

5. The welding helmet of claim 1, wherein said image includes an image of said welding operation.

6. The welding helmet of claim 1, wherein said image is displayed with a first size during said welding operation and said image is displayed at a second size, which is different than said first size, when said welding operation is not occurring.

7. The welding helmet of claim 1, wherein said helmet further includes an audio sensing device to detect audio instructions from a user of said helmet.

8. The welding helmet of claim 1, wherein said visual display portion allows said welding operation to be viewed through at least a portion of said visual display portion during said welding operation.

9. The welding helmet of claim 1, further comprising a second visual display portion which is coupled to the main body portion and is separate from said visual display portion, where said second visual display portion also allows said welding operation to be viewed with said second display portion.

10. The welding helmet of claim 9, wherein each of said visual display portion and said second visual display portion are coupled to first and second cameras, respectively, which are secured to an outer surface of said main body portion, where said visual display portion and said second visual display portion display images from said first and second cameras, respectively.

11. A welding system; comprising:
a welding power supply system which supplies a welding power to a welding torch for welding a work piece; and
a welding helmet to be worn by a welder during a welding operation with said welding power supply system, where said welding helmet is in communication with said welding power supply system, and said welding helmet comprises:
a main body portion;
a visual display portion which is coupled to the main body portion, where said display portion allows a welding operation to be viewed with said display portion;
an information generating mechanism which generates an image to be displayed on said visual display portion said that said image is viewable during said welding operation and where said image displays setting information from said welding power supply system; and
a wireless communication device coupled to said information generating mechanism which receives information to be displayed in said image;
wherein said image is generated by said information generating mechanism is generated having a visual focal range which is located beyond said visual display portion at a position outside of said main body portion and wherein said focal range is correlated to a work area for said welding operation.

12. The welding system of claim 11, further comprising a camera mounted to an outside of said main body portion of said helmet so that said camera can view the work area and sense a distance to said work area and said camera is used to calibrate said focal range of said image.

13. The welding system of claim 11, further comprising a camera mounted to an outside of said main body portion so that said camera can view the work area and said camera is used to record a welding operation in said work area.

14. The welding system of claim 11, wherein said information generating mechanism is wirelessly coupled to said welding power supply system using said wireless communication device so that information from said welding power supply system can be communicated to said helmet.

15. The welding system of claim 11, wherein said image includes an image of said welding operation.

16. The welding system of claim 11, wherein said image is displayed with a first size during said welding operation and said image is displayed at a second size, which is different than said first size, when said welding operation is not occurring.

17. The welding system of claim 11, wherein said helmet further includes an audio sensing device to detect audio instructions from a user of said helmet.

18. The welding system of claim 11, wherein said visual display portion allows said welding operation to be viewed through at least a portion of said visual display portion during said welding operation.

19. The welding system of claim 11, further comprising a second visual display portion which is coupled to the main body portion and is separate from said visual display portion, where said second visual display portion also allows said welding operation to be viewed with said second display portion.

20. The welding system of claim 19, wherein each of said visual display portion and said second visual display portion are coupled to first and second cameras, respectively, which are secured to an outer surface of said main body portion, where said visual display portion and said second visual display portion display images from said first and second cameras, respectively.

21. The welding system of claim 17, wherein said welding power supply system changes at least one welding parameter based upon said audio instructions.

22. The welding system of claim 1, wherein said welding torch has at least one sensor which can detect a working distance between said helmet and said welding operation and said detected distance is used to determine said visual focal range for said image.
